(19) United States
(12) Patent Application Publication
(10) Pub. No.: US 2014/0161891 A1
(43) Pub. Date: Jun. 12, 2014

(54) COMPOSITION OF PI3K INHIBITOR AND USE THEREOF

(71) Applicants: WU-CHOU SU, Tainan City (TW); DAR-BIN SHIEH, Tainan City (TW); CHEN-SHENG YEH, Tainan City (TW); CHIA-CHENG HOU, Kaohsiung City (TW)

(72) Inventors: WU-CHOU SU, Tainan City (TW); DAR-BIN SHIEH, Tainan City (TW); CHEN-SHENG YEH, Tainan City (TW); CHIA-CHENG HOU, Kaohsiung City (TW)

(21) Appl. No.: 13/710,026

(22) Filed: Dec. 10, 2012

(51) Int. Cl. A61K 9/50; A61K 31/5377

(52) U.S. Cl. CPC A61K 9/5031; A61K 31/5377
USPC 424/497; 514/233.5

(57) ABSTRACT

The present invention is related to a composition of PI3K inhibitor, comprising: 0.01˜10 mg of PI3K inhibitor; 10˜500 mg of poly(lactic-co-glycolic acid) (PLGA) which is encapsulated onto the surface of the PI3K inhibitor and the surface is non-modified by a modifier; and the composition has a size of 10˜1000 nm. Thereby, an excellent effect on suppressing the growth of tumor cells will be achieved by the encapsulation of PI3K inhibitor into PLGA nanomaterials without any modifier on its surface, the optimization of a ratio of PI3K inhibitor to PLGA, and the accordingly slow release of the composition.

### BACKGROUND OF THE INVENTION

1. Field of the Invention

The present invention relates to a composition of PI3K inhibitor and a use thereof, and more particularly to the composition of PI3K inhibitor encapsulated on the surface of poly(lactic-co-glycolic acid) (PLGA) nanomaterials without being modified by a modifier.

2. Description of Related Art

Current researches discovered that Phosphoinositide 3-kinase pathway (PI3K pathway) is closely related to the occurrence of several types of human tumors such as breast cancer, lung cancer, melanoma and lymphoma. Therefore, its inhibitor −PI3K inhibitor plays an important role in the research of tumor resisting drugs (including cytotoxic chemotherapy drugs and targeted drug).

2-(4-Morpholinyl)-8-phenyl-4H-1-benzopyran-4-one (LY294002) is one of the common PI3K inhibitors, which is a derivative of quercetin connected to morpholine. Therefore, LY294002 can be considered as a derivative of quercetin with the effect of suppressing the growth of cells and promoting cell apoptosis. However, LY294002 is highly toxic, and thus using LY294002 along will incur harms to human bodies to a certain extent.

In addition, poly-lactide-co-glycolide (PLGA) is a non-toxic polymer with high biocompatibility and biodegradability. At present, PLGA is developed for applications in the field of tissue engineering, biomedical engineering or drug carriers extensively. After PLGA is made into PLGA nanoparticles, the original hydrophobic property of PLGA will be changed to the hydrophilic property, so that PLGA nanoparticles will have a high degree of dispersion in water solution and become polymer nanomaterials of carrying drugs, and PLGA can be used more extensively in system with water solution in living organisms. To safely and effectively apply PLGA in living organisms, the diameter and the uniformity of the diameter of PLGA nanoparticles must be controlled during the manufacturing process. In a general manufacturing method, polyvinyl alcohol (PVA) or an equivalent polymer is generally added to serve as a stabilizer to stabilize PLGA nanoparticles in order to dissolve PLGA nanoparticles in a solvent stably. However, once if the surface of the PLGA nanoparticles is encapsulated by polyvinyl alcohol, the polymer stabilizer will not longer have functional groups, so that it is difficult to modify the surface chemically. As a result, the connection of functional biological molecules onto the surface of the PLGA nanoparticles will be restricted.

### SUMMARY OF THE INVENTION

In view of the drawbacks of the prior art, it is a primary objective of the invention to provide a composition of PI3K inhibitor, wherein the PI3K inhibitor is encapsulated on the surface of poly(lactic-co-glycolic acid) (PLGA) nanomaterials without being modified by a modifier. Since the surface of the PLGA nanomaterials is not coated with any polymer, therefore an internal carrying drug can be manufactured, and the composition providing functional surfaces of the drug is advantageous to the development of new medicines and cancer treatments.

Another objective of the present invention is to provide a composition of PI3K inhibitor, wherein the ratio of the PI3K inhibitor and PLGA is optimized, so that the composition of the present invention has the effect of suppressing tumors to approximately 250˜500 times better than the conventional nanomaterials of PLGA modified by a modifier or LY294002 without any encapsulated nanomaterials.

A further objective of the present invention is to provide a composition of PI3K inhibitor for suppressing tumors, and the composition is released slowly, and only a small quantity of the concentration can achieve an excellent effect of suppressing the growth of tumor cells.

To achieve the aforementioned objective, the present invention provides a composition of PI3K inhibitor, comprising: 0.01˜10 mg of PI3K inhibitor; and 10˜500 mg of poly(lactic-co-glycolic acid) (PLGA) encapsulated onto a surface of the PI3K inhibitor, wherein the surface of the poly(lactic-co-glycolic acid) (PLGA) is not modified by a modifier; and the composition has a size of 10˜1000 nm.

In a preferred embodiment, the PI3K inhibitor is LY294002. The PI3K inhibitor of the invention is not limited to LY294002 only, but any other equivalent derivative of quercetin can be used instead.

In a preferred embodiment, the PI3K inhibitor is comprised of 0.05 mg˜3 mg of LY294002. In another preferred embodiment, the PI3K inhibitor is comprised of 20 mg˜200 mg of the poly(lactic-co-glycolic acid) (PLGA). The concentrations of LY294002 and poly(lactic-co-glycolic acid) (PLGA) can be changed according to the size of the composition.

In a preferred embodiment, the poly(lactic-co-glycolic acid) (PLGA) has a viscosity of 3.5 A, 4 A or 4.5 A depending on the desired size of the composition. For example, a viscosity of 4.5 A of PLGA can be used to form a larger composition approximately equal to 70˜80 nm.

In a preferred embodiment, the composition has a size of 80˜120 nm that can prevent the immune system of living organism from being attacked.

The present invention further provides a use of the composition of PI3K inhibitor for suppressing tumors, wherein the composition has a concentration of 0.1˜10 μM.

In a preferred embodiment, the composition has a concentration of 0.25˜5 μM.

### BRIEF DESCRIPTION OF THE DRAWINGS

FIG. 1 is a schematic view of LY294002 encapsulated on the surface of poly(lactic-co-glycolic acid) (PLGA) without being modified by a modifier;

FIGS. 2A, 2B and 2C show a chart of data of the particle diameter and the polydispersity index of SF-LY NPs, a scanning electronic microscope photo of SF-LY NPs and a curve of release rate versus time of LY294002 respectively;

FIG. 3 show electrophoresis and cell activity diagrams of ionized LY, SF-NPs, SF-LY NPs in different cell strains respectively;

FIG. 4 show electrophoresis and cell activity diagrams of PVA-LY NPs indifferent cell strains respectively;

FIG. 5 is a curve showing the change of tumor size of mice injected with normal saline, SF NPs, ionized LY, and SF-LY NPs; and

FIG. 6 show curves of changes of the weight and the survival rate of mice injected with normal saline, SF NPs, ionized LY, and SF-LY NPs.

### DETAILED DESCRIPTION OF THE PREFERRED EMBODIMENTS

The technical contents and characteristics of the present invention will be apparent with the detailed description of a preferred embodiment accompanied with related drawings as follows. It is noteworthy that the drawings are provided for the purpose of illustrating the present invention, but not intended for limiting the scope of the invention.

### Example 1 of Preparation

LY294002 is encapsulated on the surface of PLGA without being modified by a modifier to form a nano-scale composition.

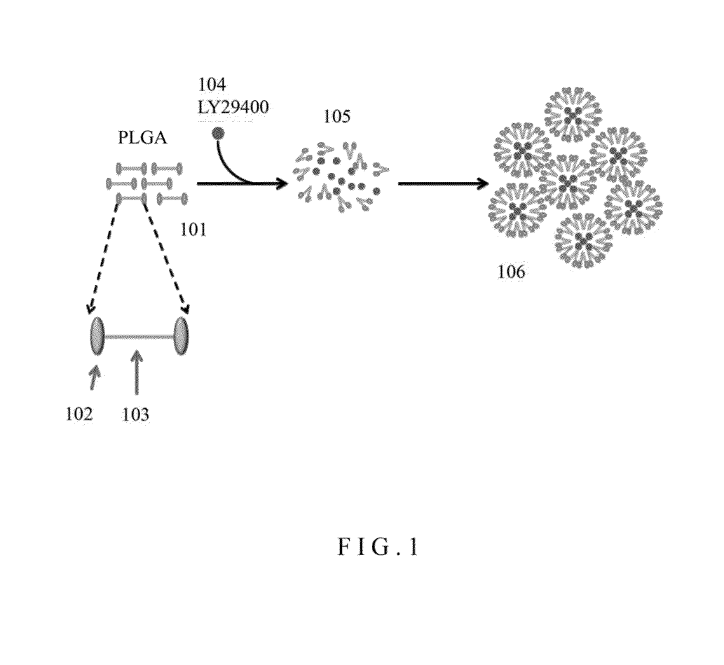

With reference to FIG. 1 for a schematic view of LY294002 encapsulated on the surface of poly(lactic-co-glycolic acid) (PLGA) powder (101) without being modified by a modifier, and the PLGA powder (101) includes a PLGA hydrophilic region (102) and a PLGA hydrophobic region (103), and LY294002 (104) with the molecular structure as shown below is added, so that the PLGA hydrophobic region (103) of the PLGA powder (101) is encapsulated on the LY294002 (104), and the semi-finished good (105) of the composition formed by LY294002 and PLGA is used for forming nanoparticles in a molding process. In other words, a finished good (106) of the composition formed by LY294002 and PLGA is produced.

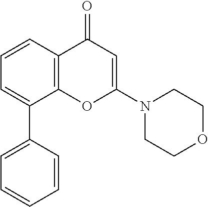 [SMILES: O=c1cc(N2CCOCC2)oc2c(-c3ccccc3)cccc12]

The detailed experiment procedure of the aforementioned manufacturing process is described as follows: PLGA polymers (SF) and LY294002 (LY) of different quantities are dissolved in 5 ml of acetone, and then a peristaltic pump is used to drop an ethanol/water (50/50%, v/v) solution into the PLGA solution slowly at a speed of 1 ml/min. The solution is blended at 240 rpm until the mixture becomes cloudy, and then the suspension is moved to 20 mL of deionized water and blended at 300 rpm for 15 minutes. The solution is placed in a suction flask for 30 minutes to remove the organic solvents. To prevent possible aggregations of the PLGA, a 90-mm filter is used to filter the solution to obtain nanoparticles. To measure the encapsulation rate of SF-LY NPs, a centrifuging method is used in this preferred embodiment to collect PLGA nanoparticles, and the PLGA nanoparticles are dissolved into acetonitrile completely. An UV-VIS spectrometer with a wavelength of 300 nm is used to measure the absorption rate.

### Example 2 of Preparation

The change of encapsulation rate is observed after the ratio of LY294002 to PLGA is changed.

Based on the preparation method as described in Example 1, the ratio of LY294002 to PLGA is changed, and then the change of encapsulation rate is observed, and the encapsulation rate is calculated by the following formula:

$$Encapsulation rate ( \% ) = \frac{Mass of drug in \text{?}}{Mass of drug in formulation} \times 100$$

$$\text{?} \text{indicates text missing or illegible when filed}$$

The results of the encapsulation rates are listed in Table 1.

TABLE 1
Encapsulation rate (%) of a nano-scale composition
in different ratios of LY294002 to PLGA

| | | | PLGA | | | |
|---|---|---|---|---|---|---|
| | | | 20 mg | 50 mg | 100 mg | 200 mg |
| LY294002 | 0.05 | mg | 89% | 99.9% | 99.9% | 99.9% |
| | 0.1 | mg | 76% | 99.9% | 99.9% | 99.9% |
| | 0.25 | mg | 65% | 99.9% | 99.9% | 99.9% |
| | 0.5 | mg | 55% | 95% | 99.9% | 99.9% |
| | 1 | mg | 25% | 89% | 95% | 95% |
| | 3 | mg | 12% | 37% | 55% | 67% |

From the results obtained by using a constant quantity of PLGA to be encapsulated on LY294002 in different ratios, we found that the more the LY294002, the smaller is the encapsulation rate. However, the drop of the encapsulation rate will reach a saturation point below a certain ratio of PLGA: LY294002. For example, 20 mg of PLGA encapsulated on LY294002 in different ratios, we found that although the encapsulation rate drops for the 0.5 mg˜3 mg of PLGA, yet the difference of the encapsulated contents is not large. In this preferred embodiment, parameters with the most appropriate ratio can be found for biological experiments. Although a larger quantity of PLGA can be used to achieve a better encapsulation rate, excessive PLGA may cause an insufficient dispersity of the solution and result in a sticky solution, or too-large PLGA nanoparticles that will be precipitate easily and cannot be dispersed uniformly.

The size of nanoparticles is approximately equal to 200˜800 nm when more than 100 mg of PLGA is encapsulated on LY294002 in different ratios. In the following experiments, approximately 50 mg of PLGA and 1 or 3 mg of LY294002 are used for conducting the experiments.

### Testing Example 1

### The Average Particle Diameter of SF-LY NPs is Detected

To measure the properties of PLGA nanoparticles (SF-LY NPs) encapsulated on LY294002, the following experiment is performed to analyze the particle diameter of SF-LY NPs in this preferred embodiment.

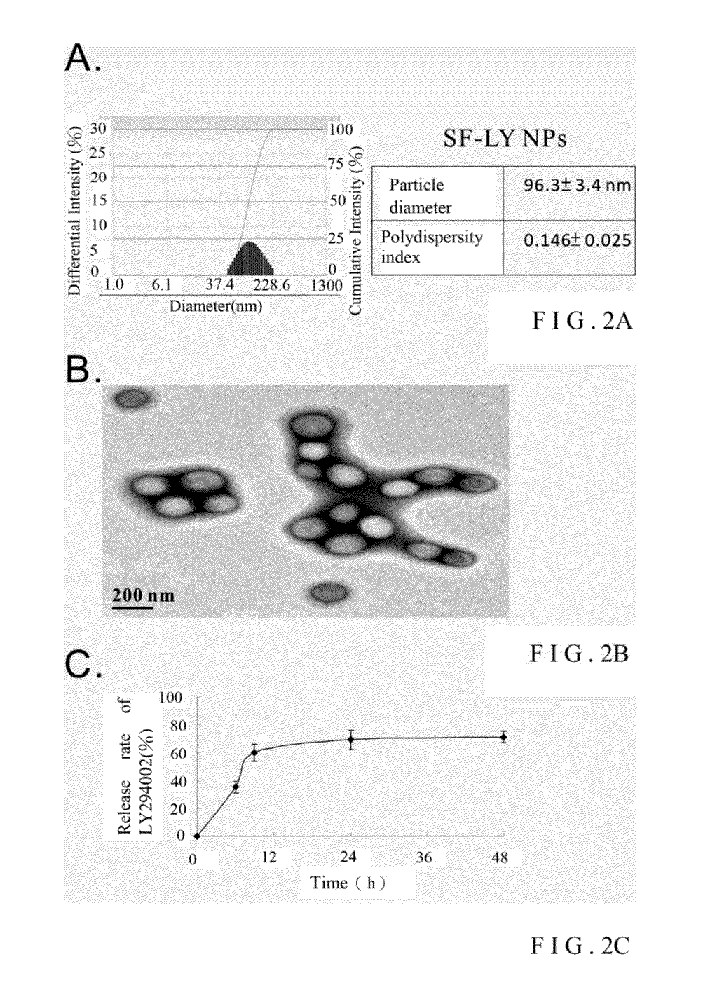

Firstly, 50 mg of PLGA powder and 3 mg of LY294002 are mixed into 5 ml of acetone, and then a peristaltic pump is used to drop an ethanol/water (50/50%, v/v) solution into the PLGA solution slowly at a speed of 1 ml/min. The solution is blended at 240 rpm until the mixture becomes cloudy, and then the suspension is moved to 20 mL of deionized water and blended at room temperature at 300 rpm for 15 minutes. The solution is placed in a suction flask for 30 minutes to remove the organic solutes. To prevent possible aggregations of the PLGA, a 90-mm filter is used to filter the solution to obtain nanoparticles. The average particle diameter is measured by a dynamic light scattering (DLS) measuring method. The results of this experiment are shown in FIG. 2A.

The nanoparticles are dropped into a copper net and vacuumed to remove moisture. The nanoparticles are stained with an 1% sodium phosphotungstate solution (pH 7.0) and developed before viewing under an electronic microscope. The results of this experiment are shown in FIG. 2B.

The nanoparticles are contained in a 1.5 ml-Eppendorf tube containing a PBS buffer solution (10 mM, pH 7.4). A suspension containing ionized LY2940025 is connected after a fixed time, and an UV-VIS spectrometer with a wavelength of 300 nm is used to measure the released LY294002. The results of this experiment are shown in FIG. 2C.

In FIG. 2A, the particle diameter is measured to be 96.33 nm by a dynamic light scattering (DLS) measuring method. In FIG. 2B, a photo taken under an electronic microscope shows that the nanoparticles are in a spherical shape, and the average particle diameter of SF-LY NPs is equal to 80 nm. In FIG. 2C, the time of releasing LY in this preferred embodiment is measured, and the curve as shown in FIG. 2C indicates that the releasing speed increases rapidly in the first 9 hours, and continues increasing for 48 hours before reaching a saturation. This phenomenon shows a continuously releasing effect.

The test results also show that the nano-scale composition has a size falling within a range from 80 nm to 120 nm and capable of preventing the immune system of living organisms from being attacked. If the size of the nanoparticles is too large, the nanoparticles may be accumulated in the living organisms easily, and thus there is a possibility of having vacular occulations or the immune system may recognize the nanoparticles as external foreign matters and engulf the nanoparticles by phagocytosis, and thus failing to achieve the medical effect in the living organisms. On the other hand, if the size of the nanoparticles is too small, the metabolism will be too quick, so that the nanoparticles will be discharged with excrements quickly.

For the too-large nanoparticles, PEG or another equivalent surface modifier is required to modify the nanoparticles in order to prevent attacks to the immune system. However, the PLGA nanoparticles of the present invention do not require any surface modification by using a modifier. The invention simply controls the nano size to prevent attacks to the immune system.

### Testing Example 2

### Electrophoresis and Cell Activity Test in Different Cell Strains are Conducted

I. For ionized LY, SF-NPs, and SF-LY NPs

To measure the ionized LY, PLAG nanoparticles (SF-NPs), and SF-LY NPs, PLGA NPs are applied to four selected types of lung cancer cell strains including AS2 (PTEN null), H157 (PI3KCA, PTEN null), H460 (PI3KCA) and H1650 (PTEN null) in this preferred embodiment, and different concentrations are used to process the ionized LY or SF-LY NPs and a MTT testing method is used to measure the cell activity, and the experiment procedure is described in details as follows:

(1) For the electrophoresis, cells are placed in ice for 30 minutes, and a cell lysis (containing a mixture of Tris 50mM pH 7.2˜7.8, NP-40 1%, EDTA 2 mM, NaCl 100 mM, 0.1% SDS supplementary liquid and protease prohibitor) (Roche Applied Sciences, Indianapolis, Ind., USA) for cell lysis. The lysate is collected by a centrifuge at 14000 rpm for 10 minutes, and Bradford testing method (Bio-Rad, Richmond, Calif., USA) is used to measure the protein concentration.

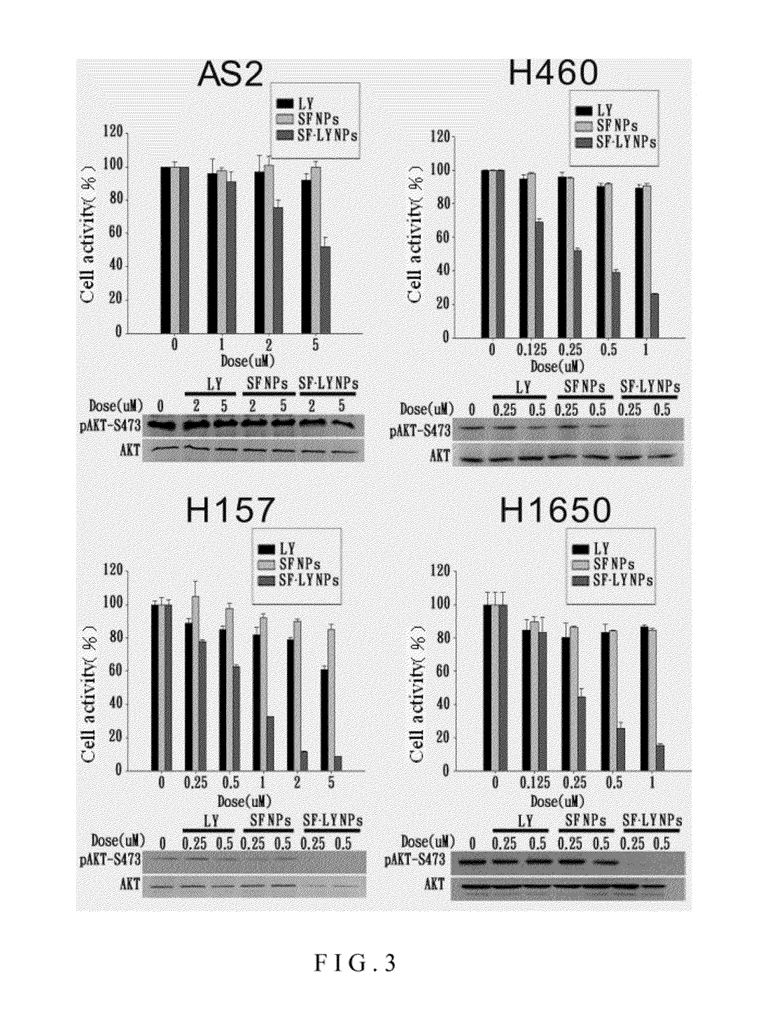

Before the protein extract is separated from SDS-PAGE, 20˜50 mg of each protein is prepared and boiled for 5 minutes. The samples are processed by gel electrophoresis for 90 minutes, and then a blotter (Amersham Pharmacia Biotech Inc., Piscataway, N.J., USA) is used to blot the samples to a PVDF film (Millipore, Billerica, Mass., USA) by 400 mA of current. The PVDF film is shaken by using skim milk (5% in TBST) at room temperature for 6.0 minutes, and then the milk is washed away, and a TBST buffer solution containing antigens (such as pAKT-s473, AKT, pERK, ERK, p-4EBP1, 4EBP 1 and actin) is shaken uniformly at 4□ till the next day, and horseradish peroxidase-conjugated secondary antibodies are shaken at room temperature for 60 minutes. After the secondary antibodies are washed away, an ECL kit (Amersham) is used to perform a luminescence test according to the instructions given by the manufacturer's manual. The results of this test are shown in FIG. 3.

(2) Cell Activity Test:

The cells are inoculated in 96-hole culture boards (each hole has 5×10³ cells) and cultured by 5% of CO₂ at 37□ till the next day. In four types of different cells, different doses of SF NPs, LY or SF-LY NPs are added and processed for 48 hours, and then a stock solution (with a content of 5 mg/ml in PBS) of a MTT agent at 37° C. is added to the 96-hole culture boards containing different processing drugs and wait for 4 fours before centrifuging the culture boards at 1200 rpm for 5 minutes, and after adding DMSO (capable of dissolving and precipitating products produced by the reaction of MTT and cells) for 5 minutes, the suspension is moved to a new ELISA board, and an ELISA reader (Varioskan, Thermo Electron) is used to measure the light absorption by 490 nm. The results of this test are shown in FIG. 3.

(3) Results: In 48 hours after the culture takes place, the lung cancer cells have no significant toxicity under the treatment of SF NPs. The ionized LY group shows a slight toxicity of the cells. For a higher dosage, a slight toxicity to H157 cells is shown. On the contrary, observations show that SF-LY NPs has significant cytotoxic effect on the three types of cell strains (H460, H157 and H1650) with a concentration falling within a range of 0.5˜1 Mm. In FIG. 3, the AS2 cells have a significant cytotoxic effect of the SF-LY NPs at a higher concentration. In this preferred embodiment, a western blotting can be used for monitoring and measuring the phosphorylated AKT content in 473 serine residue and quantifying the activated pAKT/AKT content. Undoubtedly, the SF-LY NPs group as shown in FIG. 3 shows a significant drop of the overall activated AKT content. With the same concentration, the data of this preferred embodiment show that SF-LY NPs can increase the cytotoxicity more than the ionized LY of different concentrations. This result shows that SF-LY NPs among the four types of cell strains has a stronger suppressing effect, and only a concentration above 0.25˜5 μM is required.

2. Control

In this preferred embodiment, poly(lactic-co-glycolic acid) nanocapsules (PVA-LY NPs) can be synthesized and modified by a modifier PVA to perform the aforementioned electrophoresis and MTT assays is used to test the activity of cytotoxicity of the four types of cell strains, wherein the experiment procedures of the electrophoresis and cell activity are the same as those described above, and thus will not be repeated. The method of preparing PVA-LY NPs is described as follows:

50 mg of PLGA powder and 3 mg of LY294002 are mixed uniformly in 2.5 ml of acetone, and added into 25 ml of 2% polyacrylic acid solution, and a homogenizer is used for emulsification. Such liquid is poured into 100 ml of a liquid containing 2% polyacrylic acid solution to disperse the nanoparticles, and the liquid is stirred uniformly at room temperature for 4 hours to vaporize the organic solvents. Finally, the nanoparticles PVA-LY NPs are collected by an ultracentrifuge.

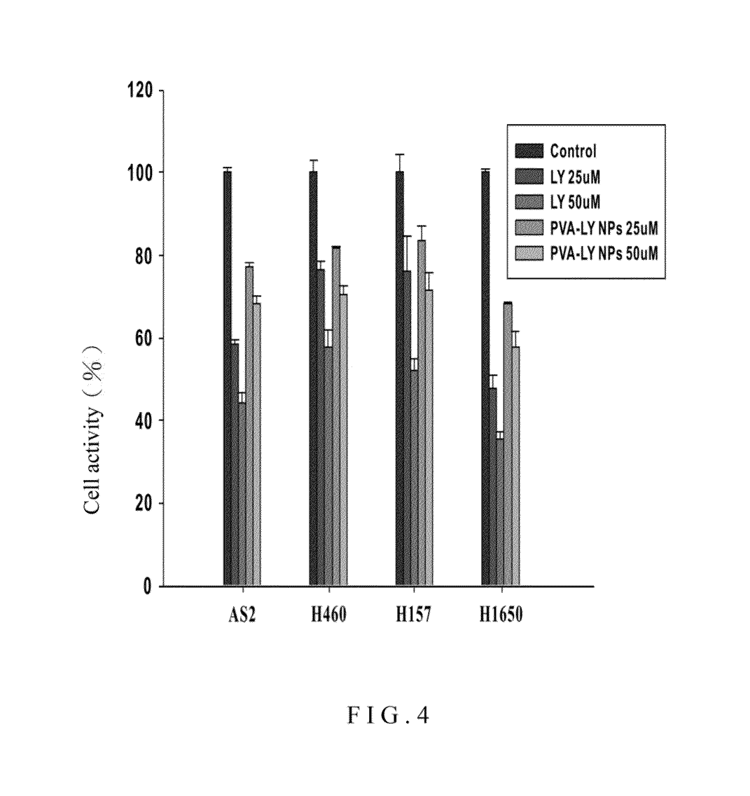

The results of this test are shown in FIG. 4, wherein PVA-LY NPs has a less effect on suppressing the growth of cancer cells, and the required concentration is as high as 25˜50 μM.

### Testing Example 3

### Animal Experiment

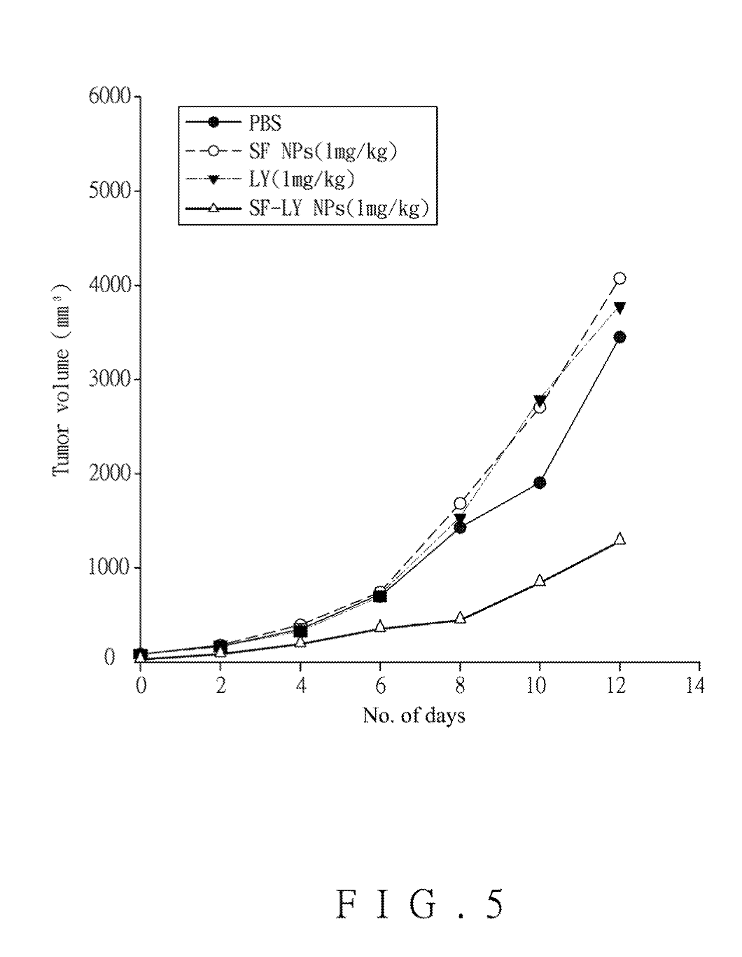

Balb/c female nude mice were obtained from National Laboratory Animal Center, Taiwan. Mice of 6˜8 weeks old are used. The mice are randomly divided into four groups, and situated in the same environment with controlled tempeature, humidity and 12 h-light/dark cycle, and the controlled environmental conditions follows the environmental conditions for animal breeding set forth by National Cheng Kung University. The balb/c immunodeficient mice are innoculated with PC14PE6/AS2 cells (1×10⁶ cells/1004 of PBS) by the subcutaneous inoculation method. If the tumor size is approximately equal to 50-60 cubic millimeters, the experiment starts taking place. According to the experiment requirements, a saline group (n=5), a PLGA nanoparticles (SF NPs) group (N=5), a LY294002 (LY) group with the same dose (1 mg/kg) (N=5) or a PLGA nanoparticles (SF-LY NPs) group encapsulated with LY294002 (N =6) are provided, and the mice are injected three times a week (Monday, Wednesday, and Friday) continuously for two weeks. The mice with the innoculations are observed in every other two days to check whether there is an abnormality. The tumor size is measured by a caliper and a standard tumor volume measurement method (Volume=Long Axis×Short Axis²×0.5). Based on human moral standards, euthanasia of the mice takes place when the tumor size reaches an average size of 4000 mm³. The results of this animal experiment are shown in FIG. 5.

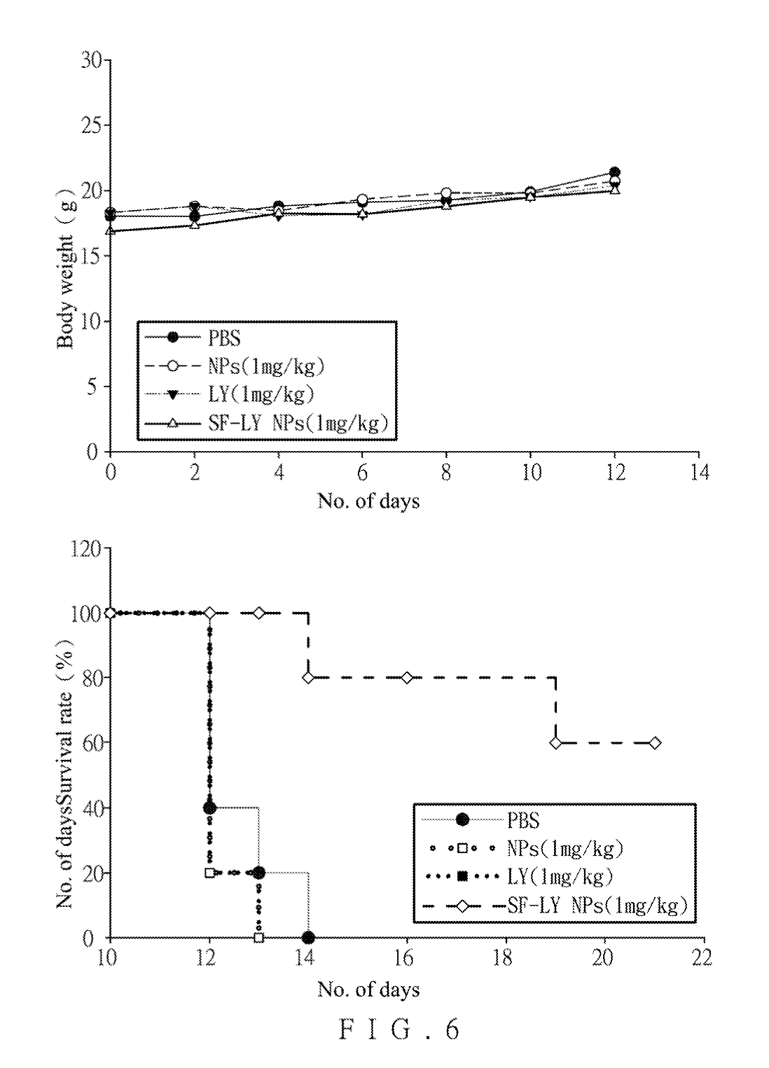

After the procedure as shown in FIG. 5 takes place, the body weight of the mice is measured in every two other days to check whether there is a loss of weight. If there is no loss of weight, then it will be considered that there is no toxicity produced in the circulatory systems. The survival rate is determined by the number of days when a natural death of each group of mice occurs or the tumor reaches a size of 4000 mm³. The results of this experiment are shown in FIG. 6.

The experiment results show that after the cells are transplanted, an AS2 tumor mode of the bald/c mice is developed in 14 days. At the beginning, the average tumor size is 50 to 60 mm³, and injections are applied in the tumor three times a week for two weeks, and then the following treatments are given: (i) Saline, (ii) LY (1 mg/kg), (iii) SF NPs (1 mg/kg) and (iv) SF-LY NPs (1 mg/kg). The change of tumor size is monitored from the beginning until the average tumor size reaches 3000˜4000 mm³ after the injections. This result shows that saline, SF NPs and ionized LY injected into the mice will increase the tumor size steadily with time. After 12˜14 days, the tumor size will be approximately 40 times bigger, indicating that the SF NPs treatments does not cause toxicity, and the ionized LY is insufficient to reduce the growth of tumors. On the other hand, the SF-LY NPs (1 mg/kg) group as shown in FIG. 5 can effectively slow down the growth of tumors and inhibit the overall volume of the tumors from increasing to approximately 2.5˜3 times of their original size. In FIG. 6, the mice of each group do not have any significant loss of weight. In FIG. 5, the time for a tumor reaching a size of 4000 mm³ is used to measure the survival rate, and the mice treated with SF-LY NPs has a significantly higher survival rate than those treated with saline, SF NPs and ionized LY. Overall speaking, these results show that the injection of SF-LY NPs into tumors induces a long-term sustainable effect of suppressing tumors.

While the invention has been described by means of specific embodiments, numerous modifications and variations could be made thereto by those skilled in the art without departing from the scope and spirit of the invention set forth in the claims.

What is claimed is:

1. A composition of PI3K inhibitor, comprising:
0.01 mg to 10 mg of PI3K inhibitor; and
10 mg to 500 mg of poly(lactic-co-glycolic acid) (PLGA) encapsulated onto the PI3K inhibitor, and surfaces of the poly(lactic-co-glycolic acid) (PLGA) being non-modified by a modifier;
wherein the composition has a size from 10 nm to 1000 nm.

2. The composition of PI3K inhibitor as recited in claim 1, wherein the PI3K inhibitor is (2-(4-Morpholinyl)-8-phenyl-4H-1-benzopyran-4-one) (LY294002).

3. The composition of PI3K inhibitor as recited in claim 1, wherein the PI3K inhibitor is comprised of 0.05 mg to 3 mg of LY294002.

4. The composition of PI3K inhibitor as recited in claim 1, wherein the poly(lactic-co-glycolic acid) (PLGA) weighs 20 mg to 200 mg.

5. The composition of PI3K inhibitor as recited in claim 1, wherein the poly(lactic-co-glycolic acid) (PLGA) has a viscosity equal to 3.5 A, 4 A or 4.5 A.

6. The composition of PI3K inhibitor as recited in claim 1, wherein the composition has a size from 80 nm to 120nm.

7. A use of a composition of PI3K inhibitor for suppressing tumors, wherein the composition has a concentration from 0.1 μM to 10 μM.

8. The use of a composition of PI3K inhibitor for suppressing tumors as recited in claim 7, wherein the composition has a concentration from 0.25 μM to 5 μM.
